(19) United States
(12) Patent Application Publication
(10) Pub. No.: US 2003/0143293 A1
(43) Pub. Date: Jul. 31, 2003

(54) Compositions and methods for treating diarrhea

(76) Inventor: Sergei Shushunov, Glencoe, IL (US)

Correspondence Address: Joseph A. Mahoney,, Mayer, Brown & Platt, P.O. Box 2828, Chicago, IL, 60690, US

(21) Appl. No.: 10/062,981

(22) Filed: Jan. 31, 2002

(51) Int. Cl. A61K 35/78; A61K 39/395; A61K 31/704; A61K 31/65; A61K 31/545; A61K 31/47

(52) U.S. Cl. 424/773; 514/37; 514/152; 514/192; 514/200; 514/312; 424/130.1

(57) ABSTRACT

This invention relates to compositions, methods, combinations, and kits for treating, preventing, or reducing the risk of developing diarrhea in a mammal; or treating, preventing, or reducing the risk of developing a symptom associated with, or related to, diarrhea in a mammal. In particular, the compositions, methods, combinations, and kits comprise an anti-diarrheal agent and an electrolyte.

### FIELD OF THE PRESENT INVENTION

This invention relates to compositions, methods, combinations, and kits for treating, preventing, or reducing the risk of developing diarrhea in a mammal; or treating, preventing, or reducing the risk of developing a symptom associated with, or related to, diarrhea in a mammal.

### BACKGROUND OF THE PRESENT INVENTION

Acute and chronic diarrhea represent a major medical problem in many areas of the world. The major medical consequences of diarrheal diseases include dehydration, acidosis, impaired growth, malnutrition, and death.

Although the major consequences of diarrheal diseases are very similar, there are numerous causes of diarrhea. Secretory and exudative diarrhea are primarily caused by bacterial or viral infections. The most common diarrheal causing bacteria is enterotoxogenic E-coli having the K99 pilus antigen. Common viral causes of diarrhea include rotavirus and coronavirus. Other infectious agents that cause diarrhea include adenovirus, cryptosporidium, shigella, cholera, vibrio bacteria, giardia lamblia, and salmonella, among others. Rotaviruses have been estimated to cause 30-50% of all cases of severe diarrheal disease in humans.

Rotavirus-induced diarrhea is classified as secretory diarrhea. Secretory diarrhea can accompany gastrointestinal disorders such as inflammatory bowel disease. Secretory diarrheas are a dangerous condition in unhealthy subjects especially in patients with acquired immunodeficiency syndrome (AIDS) and chronic inflammatory bowel disease. Diarrhea in AIDS patients can cause wasting and can be an important factor in the decline of these patients. AIDS patients often develop diarrhea due to enteric infections which their immune system is not capable of fighting off, but AIDS patients may also develop diarrhea by AIDS enteropathy. AIDS enteropathy is a disorder characterized by diarrhea without the involvement of secondary infections. It is caused by the human immunodeficiency virus (HIV) infection of the small bowel mucosal cells and colonic mucosal cells. The most common infective agent causing diarrhea due to enteric infection in AIDS patients in cryptosporidium.

In infants, rotavirus is one of the most widely-spread pathogens of acute diarrhea. Rotavirus infection is generally accompanied by fever and dehydration secondary to vomiting and diarrhea. The virus is transmitted by close contact, especially through the fecal-oral route and possibly through the respiratory route. Rotavirus is an important pathogen in day care-acquired illnesses. The virus can remain infectious on inanimate surfaces, such as toys, for several days and up to 4 hours on human hands. Worldwide, rotavirus causes more than 125 million cases of diarrhea annually in children younger than 5 years of age. In developing countries, diarrhea accounts for approximately 3.2 million annual deaths in children under 5 years of age. The mortality rate associated with rotavirus in developing countries remains unacceptably high at more than 800,000 annual deaths. Both morbidity and treatment costs of dehydration are very expensive in developing countries. In the United States alone, an estimated $1.1 billion is spent annually to treat pediatric diarrhea. Approximately one-half of this total accounts for missed work by caregivers who seek medical treatment for the child.

Outbreaks of infectious gastroenteritis in adults has been attributed to two main groups of pathogens: (i) Calicivirus, the sole genus of the family Caliciviridae, which species include the vesicular exanthema virus of swine and related virus of cats and sea lions, and (ii) “small round viruses.” Also, sixteen million travelers to developing countries from industrialized nations every year develop diarrhea, with the severity and number of cases of diarrhea varying depending on the country and area of travel.

Diarrhea often also develops as a side effect during clinical treatment with chemotherapeutic agents. Diarrhea is most commonly associated with chemotherapeutic agents such as 5-fluorouracil, cisplatin or methotrexate. Diarrhea poses a problematic symptom for patients, and because it may provoke reductions in doses or the frequency of drug administration, diarrhea may compromise the therapeutic efficacy of chemotherapy.

Diarrhea in animals and pets such as cows, pigs and horses, sheep, goats, cats and dogs, also known as scours, is a major cause of death in these animals. Diarrhea can result from any major transition, such as weaning or physical movement. One form of diarrhea is characterized by diarrhea in response to a bacterial or viral infection and generally occurs within the first few hours of the animal's life. Infections with rotavirus and coronavirus are common in newborn calves and pigs. Rotavirus infection often occurs within 12 hours of birth. Symptoms of rotaviral infection include excretion of watery feces, dehydration and weakness. Coronavirus which causes a more severe illness in the newborn animals, has a higher mortality rate than rotaviral infection. Often, however, a young animal may be infected with more than one virus or with a combination of viral and bacterial microorganisms at one time. This dramatically increases the severity of the disease.

There are generally two groups of anti-diarrheal medications. The first group is astringents. The second group is opium derivatives. While such medications have met with some degree of success, it is an alarming fact that drug development specifically targeting diarrheal disease has been, until recently, almost nonexistent.

Treatment of diarrhea also includes rehydration therapy. Generally, oral rehydration aids in the prevention and treatment of dehydration, and consequently, the introduction of oral rehydration significantly reduces morbidity and mortality. However, oral rehydration does not reduce the volume or duration of diarrhea.

Other preparations used in the treatment of diarrhea may include probiotics (lactobacillus species); immunglobulins administered orally; as well as antiperistaltic and antisecretory agents, such as different preparations of astringents (e.g., tannins), bismuth subsalicylate, cholestyramine, anticholinergics (e.g., atropine, hyoscyamine, and metoclopramide), and loperamide. The use of such drugs in the reduction and severity of diarrhea has not proved to be consistently effective and may cause serious side effects in children, including lethargy, seizures, ileus, and respiratory depression. Loperamide, one of the most commonly used antiperistaltic agents, is not recommended for use in young children and infants. Although antiperistaltic and antisecretory agents diminish the volume of stool, shorten the duration of diarrhea, reduce the degree of dehydration, currently available agents are not feasible in the treatment of dehydration and reduction of mortality in developing countries.

A remedy of treatment for diarrhea long used in Russian folk medicine is tormentil root extract (Potentilla tormentilla), from the family Rosaceae. To date, no clinical studies have been conducted in using tormentil root extract to treat infants with rotavirus-induced diarrhea.

The treatment for diarrhea also depends on the patient and the infection source. Diarrhea which is found in travelers to industrialized nations (traveler's diarrhea) frequently is caused by bacterial pathogens which are acquired through ingestion of fecally contaminated food and/or water. Approximately 50-75% of these cases are attributed to enterotoxogenic E-coli. Although traveler's diarrhea is painful, it is generally not life-threatening and often the symptoms last only three to five days. The symptoms include urgent diarrhea, abdominal cramps, nausea and fever.

The most effective course of treatment for traveler's diarrhea is the administration of antibiotics in conjunction with oral rehydration. It has been shown that prophylactic administration of antibiotics drastically reduces the number of travelers experiencing symptoms of diarrhea. However, routine administration of antibiotics is not suggested as it may cause the development of resistant strains of bacteria. Other treatment methods include administration of bismuth subsalicylate, often taken in the form of Pepto-Bismol®, diphenoxylate and loperamide.

No specific treatment for rotaviral infection is currently available, and vaccination is still experimental. Current treatment is aimed at prevention and/or treatment of dehydration by oral or intravenous fluids and electrolytes. Prevention of rotaviral-induced diarrhea by immunizing the population at risk is not possible, since the rotavirus vaccine (RotaShield, Wyeth Laboratories, Marietta, Pa.) has been removed from the market after reports of increased incidence of intussusception after immunization. Thus, the management and prevention of dehydration is an important strategy in the treatment of rotaviral-induced diarrhea. However, in view of the major role rotaviral-induced diarrhea plays in the cause of childhood death, the World Health Organization has waged an intensive campaign to develop a safe vaccine against rotavirus infection and has stressed the importance of oral rehydration solutions to treat dehydration. The experimental vaccines that have been used include, for example, animal strains, attenuated human strains, animal-human recombinant strains, and have been designed to cover the four main human pathogenic strains.

The use of antisecretory medications, such as loperamide, to treat rotaviral-induced diarrhea have been associated with serious side effects in children, and therefore, are not feasible in this young population. Bismuth subsalicylate have been reported to decrease the amount of stool output and requirements for oral rehydration solutions in children; however, administration of bismuth subsalicylate to children may be associated with development of Reye's syndrome, and therefore is considered to be unsafe.

Thus, the main goal of rotaviral-induced diarrhea therapy remains prevention of severe dehydration, rather than treating or preventing the rotavirus infection. However, the only accepted therapeutic modality in the prevention of dehydration is use of either oral, or parenteral rehydration solutions. Many children with rotaviral-induced diarrhea, however, are unable to sustain sufficient oral fluid intake and consequently develop dehydration.

An ideal therapeutic agent for the treatment of diarrhea of any etiology should have a high index of safety even when used without a systematic control, must be compatible with oral rehydration solutions, be effective and be inexpensive.

While there is a substantial need for development of an agent for treating and preventing diarrhea, particularly diarrhea caused by rotaviral infection, no definite and efficacious method for preventing diarrhea has been identified. Quick resolution or prevention of diarrhea is important, not only to prevent hospitalization, but also to improve quality of life.

### SUMMARY OF THE INVENTION

The present invention is directed to compositions, methods, combinations, and kits for treating, preventing, or reducing the risk of developing diarrhea, for example, rotavirus-induced diarrhea, in a mammal in need thereof, or treating, preventing, or reducing the risk of developing a symptom associated with, or related to, diarrhea in a mammal in need thereof. The present invention includes methods of halting or slowing the progression of diarrhea in a mammal, for example, rotavirus-induced diarrhea, once it becomes clinically evident, or treating a symptom related to, or associated with, diarrhea, such as dehydration. The mammal may already have diarrhea at the time of administration, or be at risk of developing diarrhea. The compositions, methods, combinations, and kits of the present invention are pharmaceutical compositions comprising an anti-diarrheal agent, including, for example, a tormentil root extract, and at least one electrolyte, including, for example, sodium or potassium, where the individual agents together make an anti-diarrhea effective amount. Glucose (or dextrose) can optionally be added. The kits also contain instructions for the patient.

### BRIEF DESCRIPTION OF DRAWINGS

FIG. 1 is a graph showing a comparison of clinical outcomes between the control groups and the tormentil root extract treatment group in forty children diagnosed with rotaviral-induced diarrhea and ranging in age from 3 months to 7 years.

### DETAILED DESCRIPTION OF THE PRESENT INVENTION

While the present invention may be embodied in many different forms, several specific embodiments are discussed herein with the understanding that the present disclosure is to be considered only as an exemplification of the principles of the present invention, and it is not intended to limit the present invention to the embodiments illustrated.

Where the present invention is illustrated herein with particular reference to rotaviral-induced diarrhea, it will be understood that any diarrhea, and concomitant dehydration, can be treated using the compositions, methods and combinations provided herein. Where the present invention is illustrated herein with particular reference to tormentil root extract, it will be understood that any other anti-diarrheal or rotavirus-inhibiting agent can, if desired, be substituted in whole or in part for the tormentil root extract in the compositions, methods, combinations and kits herein described.

Besides being useful for human treatment, the present invention is also useful for veterinary treatment of companion mammals, exotic animals and domesticated animals, including mammals, rodents, and the like. In one embodiment, the mammals include horses, dogs, and cats. In another embodiment of the present invention, the human is an adolescent or infant under the age of eighteen years of age.

In the compositions, methods, combinations, and kits of the present invention, the anti-diarrheal agent, for example, a tormentil root extract, and the electrolyte are administered in an anti-diarrhea effective amount. When administered as part of a combination therapy, the anti-diarrheal agent together with the electrolyte(s) provide enhanced treatment options for treating or preventing rotavirus-induced diarrhea, or diarrhea or any other etiology, in a mammal as compared to administration of either a rotavirus-inhibiting agent or an electrolyte alone.

The term “diarrhea,” as used herein, indicates a medical syndrome which is characterized by the symptoms of diarrhea (or scours in animals). In general, diarrhea is a disorder resulting in a secretory imbalance. Diarrhea is divided into three categories based on the underlying mechanism: exudative, decreased absorption, and secretory. Exudative diarrheas result from inflammatory processes leading to impaired colonic absorption, and outpouring of cells and colloid caused by such disorders as ulcerative colitis, shigellosis, and amebiasis. Disorders of decreased absorption include osmotic, anatomic derangement, and motility disorders. Osmotic diarrhea can occur as a result of digestive abnormalities such as lactose intolerance. Anatomic derangement results in a decreased absorption surface caused by such procedures as subtotal colectomy and gastrocolic fistula. Motility disorders result from decreased contact time resulting from such diseases as hyperthyroidism and irritable bowel syndrome. Secretory diarrhea is characterized by the hypersecretion of fluid and electrolytes from the cells of the intestinal wall. In classical form, the hypersecretion is due to changes which are independent of the permeability, absorptive capacity and exogenously generated osmotic gradients within the intestine. However, all forms of diarrhea can manifest a secretory component.

Contemplated compositions, methods, combinations, and kits of the present invention are useful to treat, prevent, or reduce the risk of developing a variety of diarrheal diseases in a mammal, or the symptoms associated with, or related to a diarrheal disease. Such diarrheal disease, include, but are not limited to, exudative form of diarrhea; nonexudative form of diarrhea; decreased absorption form of diarrhea; non-decreased absorption form of diarrhea; secretory form of diarrhea; nonsecretory form of diarrhea; inflammatory form of diarrhea and noninflammatory form of diarrhea.

In an embodiment of the present invention, the compositions, methods, combinations, and kits are useful in treating secretory diarrhea induced by rotaviral infection. Rotaviral infection can be caused by any viral member of the genus Rotavirus in the family Reoviridae. Rotavirus makes up a group of RNA viruses that include, for example, human gastroenteritis virus, Nebraska calf scours virus, epizootic diarrhea virus of infant mice, and others. The genus Rotavirus is further classified into five groups, Groups A-E. Group A includes subtypes 1, 2, 3, and 4 that are the main human pathogens, and an additional 7 subtypes that infect various animals, including, for example, monkey, calf and mouse. Group B infects pigs and rats and has caused extensive outbreaks in China over the years. Group C infects pigs and occasionally humans, Group D infects birds, and Group E infects pigs. In humans, rotaviral diarrhea is generally classified as secretory diarrhea, which can accompany, for example, gastrointestinal disorders such as inflammatory bowel disease.

Rotaviral infection is found worldwide, and can occur in all ages, and re-infection can occur. In the tropics and developing countries, rotavirus-induced diarrhea occurs at all times of the year, but peaks in the summer. Children usually present with mild to moderate fever and vomiting early in the illness, followed by diarrhea and dehydration.

While not wishing to be bound by theory, it is believed that the rotaviral protein NSP4 works as an enterotoxin, and it is contemplated that certain fractions in tormentil root extract interfere with enterotoxin-induced response.

Incubation of rotavirus is short, one to three days, with sudden onset of watery diarrhea, with or without vomiting. In healthy subjects, the disease is generally self-limiting and may last up to six days, but much longer in immunocompromised individuals. Upon onset of diarrhea, dehydration usually results, and can be severe and life threatening in young children and unhealthy subjects.

Detection of rotaviral infection is from the stool and can be detected and diagnosed by those skilled in the art using such techniques as latex agglutination, ELISA, electron microscopy, and electrophoresis of RNA segments.

In another embodiment, the compositions, methods, combinations, and kits of the present invention are used in combination with other treatment methods which are known in the art to treat diarrhea caused by decreased absorption or inflammation. The compositions, methods, combinations, and kits of the present invention can also be used in conjunction with compounds involved in regulating chloride ion secretion and can function alone or when used in combination with other treatment methods to decrease net fluid secretion even when this is due primarily to abnormalities in absorption or inflammation.

The compositions, methods, combinations, and kits of the present invention are useful in treating diarrhea or scours in a subject at risk of developing these disorders. Subjects at risk of developing diarrhea or scours are those subjects which have a high likelihood of exposure to the bacterial and viral microorganisms which cause these diseases or symptoms. For example, approximately one third of travelers to developing countries will develop diarrhea; infection with rotavirus is one of the leading causes of death in infants in developing countries; patients with HIV have a greater than 50% chance of developing diarrhea; cancer patients develop diarrhea as a side effect of chemotherapy; and many newborn calves and pigs develop scours; and patients with inflammatory bowel disease develop recurrent diarrhea.

The compositions, methods, combinations, and kits of the present invention are also useful in treating subjects who already exhibit the symptoms of diarrhea or scours. Once a subject has been exposed to a microorganism causing the symptoms, the subject may be treated with the compositions, methods, combinations, and kits of the present invention in order to treat, prevent, or reduce the symptoms. The symptoms of diarrhea include bowel irregularity, fecal fluid rich in sodium or potassium, fluid feces, dehydration, fever, loss of body weight, headache, anorexia, vomiting, malaise and myalgia. The symptoms of scours include a loss of body weight or failure to grow, dehydration, malodorous feces, fluid feces, feces containing pieces of partially digested milk or semisolid material, and feces of a yellow-white or gray color.

The terms “effective amount,” “anti-diarrhea effective amount,” or “rotavirus—or rotaviral-induced diarrhea effective amount” means that the concentration of the therapeutic agent of the present invention is such that results in a therapeutic level of the therapeutic agent delivered to a subject over the term that the drug is to be used. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of menopause in accordance with the present invention. In terms of treatment protocols, it should be appreciated that the dosage to be administered will depend on several factors, including the particular agent that is administered, the route administered the condition of the particular patient, etc. Generally speaking, one will desire to administer an amount of the compound that is effective to achieve a serum level commensurate with the concentrations found to be effective in vitro. Thus, where an compound is found to demonstrate in vitro activity at, for example, 10 ng/ml, one will desire to administer an amount of the drug that is effective to provide about a 10 ng/ml concentration in vivo. Determination of these parameters is well within the skill of the art. These considerations, as well as effective formulations and administration procedures are well known in the art and are described in standard textbooks.

The term “anti-diarrheal agent” means an agent that is effective in treating diarrhea, for example one that weakens or abolishes the action of the rotavirus. In particular, an anti-diarrheal agent is efficacious at treating or preventing diarrhea of any etiology such as, for example, a rotaviral infection in a mammal, or relieving to some extent at least one of the symptoms related to, or associated with, a rotaviral infection. An anti-diarrheal agent is also efficacious in, for example, 1) reducing in the number of rotaviral particles in a subject; 2) inhibiting (i.e., slowing to some extent, preferably stopping) rotaviral cell infection; and/or 3) inhibiting to some extent rotaviral growth. These considerations, as well as effective procedures of determining and measuring the efficaciousness of an anti-diarrheal agent are well known in the art and are described in standard textbooks. Anti-diarrheal agents of the present invention include tormentil root extract, Lomotil® (diphenoxylate HCL and atropine sulfate), Levsin® (hyoscyamine), Reglan® (metoclopramide), Imodium® (loperamide), and Kaolin-Pectin (Kaopectate®).

The term “prevention,” in relation to diarrhea-induced rotaviral infection, means no diarrheal event if none had occurred, or no further diarrheal event if there had already been a diarrheal event.

The term “electrolyte” means any compound that conducts electricity while in solution and is decomposed (electrolyzed) by it. Such compounds are ionizable in solution. Examples of an electrolyte that may be used in the present invention include, for example, inorganic acids, bases, and salts. In one embodiment of the present invention, the electrolyte is, for example calcium, sodium, potassium, chloride, or phosphorus salts. In one embodiment, the calcium is present as an amino acid chelate having a ligand to calcium mole ratio of at least 1:1. In another embodiment of the present invention, the sodium and potassium are present as amino acid complexes having a ligand to metal mole ratio of 1:1, or they can be present as inorganic salts in the form of chlorides, phosphates, and the like. In yet another embodiment of the present invention, the sodium, potassium, and calcium may be present in their acetate or lactate form. Additional examples of electrolytes and electrolyte solutions that may be used in the present invention are described in Remington's The Science and Practice of Pharmacy, Meade Publishing Co., and United States Pharmacopeia/National Formulary.

The composition comprises an anti-diarrheal agent and an electrolyte(s), where the amount of the anti-diarrheal agent and the electrolyte(s) together make an anti-diarrhea effective amount. In one embodiment of the present invention, the anti-diarrheal agent comprises a solution or suspension of a tormentil root extract of about 0.01% to about 5.0% weight to weight of the composition, and an oral rehydration solution of electrolytes and dextrose as follows:

Formula 1

| Compound | Amount in 1000 ml | |
|---|---|---|
| Tormentil root extract | 50 | mg |
| Sodium | 75 | mEq |
| Potassium | 20 | mEq |
| Chloride | 65 | mEq |
| Citrate (source of bicarbonate) | 30 | mEq |
| Dextrose | 25 | g |

For example, in a 10 kg, one-year old patient, Formula 1 is dosed as follows: 20 ml/kg/dose (200 ml/dose) every 3-4 hours up to 6 doses per 24 hours depending on the severity of the diarrhea. Formula 1 can vary widely and may contain any amount from about 5 mg to about 500 mg of root extract per liter.

In one embodiment of the present invention, dosage levels of a tormentil root extract (or the constituent chemicals of the tormentil root extract) on the order of about 1.0 to about 200 mg/kg/day in single or divided doses are useful in the treatment of the above conditions. In one embodiment, the tormentil root extract comprises at least about 40% of polyphenols as a standardized extract.

The tormentil root extract is effective over a wide dosage range. For examples, dosages per day normally fall within the range of about 0.1 mg to about 10.0 mg/kg of body weight per dose up to 6 doses per day. In one embodiment, the dose is 1 mg/kg of the root extract per dose. However, it will be understood that the amount of the compound actually administered will be determined by a physician, in the light of the relevant circumstances, including the condition to be treated, the chosen route of administration, the actual compound administered, the age, weight, and response of the individual patient, the severity of the patient's symptoms, etc., and therefore the above dosage ranges are not intended to limit the scope of the present invention in any way. In some instances dosage levels below the lower limit of the aforesaid range may be more than adequate, while in other cases still larger doses may be employed without causing any harmful side effect, provided that such larger doses are first divided into several smaller doses for administration throughout the day.

Certain dosage forms of the invention will contain from about 0.1 mg to about 1000 mg of the tormentil root extract, or the active constituent chemicals of the tormentil root extract, or the equivalent per dosage unit. The formulations may contain for example, about 0.1, 10.0, 100.0, 1,000 or 10,000 mg tormentil root extract (or the active ingredient of the tormentil root extract) dosage unit.

In one embodiment of the present invention, dosage levels of an electrolyte on the order of about 0.1 mg to about 1000 mg per dosage unit are useful in the treatment of the above conditions. In another embodiment, Formula 1 may take the form of a concentrated bulk powder, which is mixed with water to form a solution or suspension before oral administration. Flavorings may also be added to the powder, liquid and chewable form to enhance the taste of the composition.

The method of the present invention comprises administering to the mammal in a combination therapy an amount of an anti-diarrheal agent, for example, a tormentil root extract, and at least one electrolyte as described above. The phrase “combination therapy” embraces the administration of an anti-diarrheal agent and at least one electrolyte as part of a specific treatment regimen intended to provide a beneficial effect from the co-action of these therapeutic agents for the treatment of diarrhea. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, weeks, or months depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule, tablet or solution having a fixed ratio of each therapeutic agent or in multiple, single capsules, tablets, or solutions for each of the therapeutic agents. Sequential or substantially simultaneous administration of each therapeutic agent can be effected by any appropriate route including, but not limited to, oral routes, percutaneous routes, intravenous routes, intramuscular routes, and direct absorption through mucous membrane tissues. The therapeutic agents can be administered by the same route or by different routes. For example, a first therapeutic agent of the combination selected may be administered orally, while the other therapeutic agent of the combination may be administered percutaneously. Alternatively, for example, all therapeutic agents may be administered orally, or all therapeutic agents may be administered percutaneously, or all therapeutic agents may be administered intravenously, or all therapeutic agents may be administered intramuscularly, or all therapeutic agents can be administered by direct absorption through mucous membrane tissues. The sequence in which the therapeutic agents are administered is not narrowly critical. “Combination therapy” also can embrace the administration of the therapeutic agents as described above in further combination with other biologically active ingredients or therapies, such as, but not limited to, probiotics (lactobacillus species); immunglobulins for example; as well as antiperistaltic and antisecretory agents, such as different preparations of tannins, bismuth subsalicylate, cholestyramine and loperamide; an immunoglobulin preparation from bovine, sheep, goat, or other mammal's colostrum; Lomotil®; an intravenous or oral rehydration fluid; a dry rehydration composition salt; an antibiotic such as tetracycline, doxycycline, penicillins, cephalosporins, aminoglycosides, trirmethoprim, sulfamethoxazole; a quinolone drug such as norfloxacin or ciprofloxacin; bismuth subsalicylate; diphenoxylate; and loperamide.

In another embodiment of the present invention, the composition is a dry preparation of the therapeutic agents of the present invention and an anti-diarrheal agent. An anti-diarrheal agent can include, for example, probiotics (lactobacillus species); immunoglobulins administered orally; as well as antiperistaltic and antisecretory agents, such as different preparations of tannins, bismuth subsalicylate, cholestyramine and loperamide (Imodium®). The dry preparation may be administered directly or may be hydrated and/or diluted in a liquid solution prior to administration. In another embodiment the composition is a liquid solution of a therapeutic agent of the present invention and an anti-diarrheal agent.

The compositions, methods, combinations, and kits can also be used with an agent to control electrolyte transport, and particularly chloride secretion. While not wishing to be bound by theory, it is believed in chloride secretion, chloride enters the cell across the basolateral membrane on a cotransporter that is coupled to entry of sodium ions (Na⁺) and potassium ions (K⁺). The entry step is electrically neutral because the charge on the anion is balanced by the charges on the cations. The cotransporter accumulates chloride ions (Cl⁻) in the cell at a value greater than that predicted for electrochemical equilibrium. Removal of sodium ions from the submucosal solution or addition of a loop diuretic (furosemide or bumetanide) inhibits chloride ions accumulation in the cell, thereby inhibiting chloride ion secretion. Na-K-ATPase in the basolateral membrane maintains the sodium ion concentration within the cell lower than that in the submucosal solution; that gradient across the basolateral membrane provides the energy required to pull chloride ions and potassium ions into the cell. As the pump hydrolyzes adenosine triphosphate, it drives sodium ions out of the cell and potassium ions into the cell; the pump maintains a low intracellular sodium ion concentration (approximately 20 mmol/L) and a high intracellular potassium ion concentration (approximately 150 mmol/L). Thus, by maintaining a low intracellular sodium ion concentration, the Na-K-ATPase provides the energy for both chloride ion secretion and sodium ion absorption. Although the activity of the sodium ion pump is required for transepithelial transport, that activity dose not directly control the rate of transport. Rather, the rate is primarily controlled by the ion channels present in both cell membranes and, possibly, by the chloride ion entry step at the basolateral membrane.

Potassium, which enters the cell on the sodium ion pump (and may also do so in the sodium ion—potassium ion—chloride ion entry step), must exit across the basolateral membrane because there is very little potassium ion secretion in most secretory epithelia. Potassium ions accumulates in the cell above electrochemical equilibrium and thus can flow passively out of the cell through basolateral potassium ion channels.

This exit of potassium ions across the basolateral membrane plays two important physiologic roles. First, it maintains a negative intracellular voltage, which is important for driving chloride ions exit across the apical membrane. Second, it prevents cell swelling, which would otherwise result from entry of potassium ions. Thus, the activity of the basolateral potassium ion channels contributes to the overall rate of transport.

In another embodiment, the compositions, methods, combinations, and kits of the present invention are used with a plant derived preparation containing tannins. For example, the composition of dry Potentilla extract consists of tannins, up to 35%; glycoside, and other components. Tannins are known to have antisecretory effect. While not wishing to be bound by any theory, the mechanism of antisecretory effect of tannins is contemplated to be through inhibition of transepitelial intestinal chloride ion secretion. In one embodiment, the tannin is administered to a subject at a dose greater than about 0.5 mg/kg/day. In another embodiment, the tannin is administered at a dose of between about 0.5 mg/kg/day to about 6,000 mg/kg/day. In another embodiment of the present invention, the tannin is administered at a dose of between about 50 mg/kg/day to about 3,000 mg/kg/day. In yet another embodiment the tannin is administered to a subject at a dose of about 600 mg/kg/day.

Antacids such as calcium carbonate, sodium bicarbonate, and magnesium and aluminum salts can be added to the compositions of the present invention in amounts effective to neutralize gastric pH.

The use of the term “about” in the present disclosure means “approximately,” and use of the term “about” indicates that dosages slightly outside the cited ranges may also be effective and safe, and such dosages are also encompassed by the scope of the present claims.

The phrase “pharmaceutically acceptable” is used adjectivally herein to mean that the modified noun is appropriate for use in a pharmaceutical product. Pharmaceutically acceptable cautions include metallic ions and organic ions. More preferred metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The therapeutic agents of the present invention are usually administered in the form of pharmaceutical compositions. These therapeutic agents can be administered by a variety of routes including oral, rectal, transdermal, subcutaneous, intravenous, intramuscular, and intranasal, as well as administration by nasogastric tube. These therapeutic agents are effective as both injectable and oral compositions. Such compositions are prepared in a manner well known in the pharmaceutical art and comprise at least one therapeutic agent. The therapeutic agents of the present invention may also be administered by other non-oral routes, including, for example, percutaneous, transmucosal, implantation, inhalation spray, rectal, vaginal, topical, buccal (for example, sublingual), or parenteral (for example, subcutaneous, intramuscular, intravenous, intramedullary and intradermal injections, or infusion techniques administration).

In one embodiment of the present invention, an electrolyte may, for example, be administered to a subject by oral routes, percutaneous routes, intravenous routes, intramuscular routes, and direct absorption through mucous membrane tissues. And may be in dry or liquid form, or as an oral or intravenous sugar-electrolyte solution or dry composition. In one embodiment, the electrolyte is administered orally to the subject as a solution. In another embodiment the electrolyte solution is administered intravenously to the subject as a solution. In yet another embodiment, the electrolyte solution is administered percutaneously to the subject as a solution. For oral administration, Rehydron® (Orion Pharma International, Finland) or Pedialyte® (Ross, USA) solution can be administrated according to the manufacturer's instructions. In yet another embodiment, a parenteral rehydration solution consists of glucose, sodium chloride and potassium chloride. In any event, administration of an electrolyte for rehydration therapy may be performed in accordance with World Health Organization recommendations. See World Health Organization, Diarrheal Diseases Control Program: A Manual For The Treatment Of Acute Diarrhea For Use By Physicians And Other Senior Health Workers. Geneva: WHO, 1984:WHO/CDD/SER/80.2(rev.1); World Health Organization; The Treatment Of Diarrhea: A Manual For Physicians And Other Senior Health Workers, http://www.who.int/chd/publications/cdd/textrev4.htm.

When administered, the therapeutic agents of the present invention are administered in pharmaceutically acceptable compositions. Such preparations may routinely contain salts, buffering agents, preservatives, compatible carriers, and optionally other therapeutic ingredients. Suitable buffering agents include: acetic acid and a salt, citric acid and a salt; boric acid and a salt; and phosphoric acid and a salt. Suitable preservatives include benzalkonium chloride; chlorobutanol; parabens and thimerosal.

The present invention also includes methods employing pharmaceutical compositions which contain, as the therapeutic agent, the compounds of the present invention associated with pharmaceutically acceptable carriers. In making the compositions of the present invention the therapeutic agent is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the therapeutic agent, soft and hard gelatin capsules, suppositories, sterile injectable solutions, and sterile packaged powders. For example, Formula 1 can take the form of frozen freezer pops.

In preparing a formulation, it may be necessary to mill the therapeutic agent to provide the appropriate particle size prior to combining with the other ingredients. If the therapeutic agent is substantially insoluble, it ordinarily is milled to a particle size of less than 200 mesh. If the therapeutic agent is substantially water soluble, the particle size is normally adjusted by milling to provide a substantially uniform distribution in the formulation, for example about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions of the present invention can be formulated so as to provide quick, sustained or delayed release of the active ingredient after administration to the patient by employing procedures known in the art.

The compositions are preferably formulated in a unit dosage form, each dosage containing from about 2.0 mg to about 500 mg, more usually about 20 mg to about 200 mg, of the active tormentil root extract ingredient. The term “unit dosage form” refers to physically discrete units suitable as unitary dosages dosages for human subjects and other mammals, each unit containing a predetermined quantity of active material calculated to produce the desired therapeutic effect, in association with a suitable pharmaceutical excipient.

For preparing solid compositions such as tablets the principal therapeutic agent is mixed with a pharmaceutical excipient to form a solid preformulation composition containing a homogeneous mixture of a therapeutic agent of the present invention. When referring to these preformulation therapeutic agents as homogeneous, it is meant that the therapeutic agent is dispersed evenly throughout the composition so that the composition may be readily subdivided into equally effective unit dosage forms such as tablets, pills and capsules. This solid preformulation is then subdivided into unit dosage forms of the type described above.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

In another embodiment of the present invention, a the therapeutic agent is formulated as a transdermal delivery device (“patches”). Such transdermal patches may be used to provide continuous or discontinuous infusion of the compounds of the present invention in controlled amounts. The construction and use of transdermal patches for the delivery of pharmaceutical agents is well known in the art. See, for example, U.S. Pat. No. 5,023,252, issued Jun. 11, 1991. Such patches may be constructed for continuous, pulsatile, or on demand delivery of pharmaceutical agents.

Injectable drug formulations include solutions, suspensions, gels, microspheres and polymeric injectables, and can comprise excipients such as solubility-altering agents (for example, ethanol, propylene glycol and sucrose) and polymers (for example, polycaprylactones and PLGA's).

Compositions suitable for parenteral administration conveniently comprise a sterile aqueous preparation of the therapeutic agent, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

Other delivery systems can include time-release, delayed release or sustained release delivery systems. Such systems can avoid repeated administrations of the therapeutic agents of the present invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di- and triglycerides; hydrogel release systems; silastic systems; peptide based systems; wax coatings, compressed tablets using conventional binders and excipients, partially fused implants and the like. Specific examples include, but are not limnited to: (a) erosional systems in which the polysaccharide is contained in a form within a matrix, found in U.S. Pat. No. 4,452,775 (Kent); U.S. Pat. No. 4,667,014 (Nestor et al.); and U.S. Pat. No. 4,748,034 and U.S. Pat. No. 5,239,660 (Leonard) and (b) diffusional systems in which an active component permeates at a controlled rate through a polymer, found in U.S. Pat. No. 3,832,253 (Higuchi et al.) and U.S. Pat. No. 3,854,480 (Zaffaroni). In addition, a pump-based hardware delivery system can be used, some of which are adapted for implantation.

Use of a long-term sustained release implant may be particularly suitable for treatment of diarrhea in immunodeficient patients, who need continuous administration of the compositions of the present invention. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above.

The compositions of the present invention may be in a unit dosage form and may be prepared by any of the methods well known in the art of pharmacy. Methods include the step of bringing the therapeutic agents into association with a carrier which constitutes one or more accessory ingredients. In general, the compositions are prepared by uniformly and intimately bringing the therapeutic agents into association with a liquid carrier, a finely divided solid carrier, or both, and then, if necessary, shaping the product.

The therapeutic agents of the present invention may also be administered to a subject in the form of a salt, ester, amide, enantiomer, isomer, tautomer, or prodrug, or derivatives of these compounds.

The following examples illustrate the pharmaceutical compositions of the present invention.

Formula 2-Unit Dose Powder

| | | |
|---|---|---|
| Tormentil root extract | 70 | mg |
| Sucrose | 200 | mg |
| Sodium Chloride | 700 | mg |
| Excipients and flavoring | 30 | mg |
| | 1000 | mg |

total per packet

This powder are placed into a foil packet, the contents of which may be mixed with about 30 ml to about 120 ml of water prior to oral administration. The powder may also be proportionately bulk compounded and placed in a large (e.g., 1 kg) container. Unit dose scoops of the bulk powder can then be mixed with water to form a solution or suspension.

Formula 3-Tablet or Capsule

| | | |
|---|---|---|
| Tormentil root extract | 70 | mg |
| Sucrose | 200 | mg |
| Sodium Chloride | 700 | mg |
| Binders and excipients | 30 | mg |
| | 1000 | mg |

total per tablet

The term “suspension tablets” as used herein refers to compressed tablets which rapidly disintegrate after they are placed in water, and are readily dispersible to form a suspension containing a precise dose of the active agents. Croscarmellose sodium is a known disintegrant for tablet formulations, and is available from FMC Corporation, Philadelphia, Pa. under the trademark Ac-Di-Sol®. It is frequently blended in compressed tableting formulations either alone or in combination with microcrystalline cellulose to achieve rapid disintegration of the tablet.

Microcrystalline cellulose, alone or co processed with other ingredients, is also a common additive for compressed tablets and is well known for its ability to improve compressibility of difficult to compress tablet materials. It is commercially available under the Avicel® trademark. Two different Avicel® products are utilized, Avicel® PH which is microcrystalline cellulose, and Avicel® AC-815, a co processed spray dried residue of microcrystalline cellulose and a calcium-sodium alginate complex in which the calcium to sodium ratio is in the range of about 0.40:1 to about 2.5:1. While AC-815 is comprised of 85% microcrystalline cellulose (MCC) and 15% of a calcium-sodium alginate complex, for purposes of the present invention this ratio may be varied from about 75% MCC to 25% alginate up to about 95% MCC to 5% alginate. Depending on the particular formulation and active ingredient, these two components may be present in approximately equal amounts or in unequal amounts, and either may comprise from about 10% to about 50% by weight of the tablet.

The suspension tablet composition may, in addition to the ingredients described above, contain other ingredients often used in pharmaceutical tablets, including flavoring agents, sweetening agents, flow aids, lubricants or other common tablet adjuvants, as will be apparent to those skilled in the art. Other disintegrants, such as crospovidone and sodium starch glycolate may be employed, although croscarmellose sodium is preferred.

In addition to the suspension tablet, the solid formulation of the present invention can be in the form of a powder, a tablet, a capsule, or other suitable solid dosage form (e.g., a pelleted form or an effervescing tablet, troche or powder), which creates the inventive solution in the presence of diluent or upon ingestion. For example, the water in the stomach secretions or water, which is used to swallow the solid dosage form, can serve as the aqueous diluent.

Dry oral formulations can contain excipients such as binders (for example, hydroxypropylmethylcellulose, polyvinyl pyrilodone, other cellulosic materials and starch), diluents (for example, lactose and other sugars, starch, dicalcium phosphate and cellulosic materials), disintegrating agents (for example, starch polymers and cellulosic materials) and lubricating agents (for example, stearates and talc).

Such solid forms can be manufactured as is well known in the art. Tablet forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmaceutically compatible carriers. The manufacturing processes may employ one, or a combination of, four established methods: (1) dry mixing; (2) direct compression; (3) milling; and (4) non-aqueous granulation. Lachman et al., The Theory and Practice of Industrial Pharmacy (1986). Such tablets may also comprise film coatings, which preferably dissolve upon oral ingestion or upon contact with diluent.

Non-limiting examples of antacid agents which could be utilized in such tablets include sodium bicarbonate, alkali earth metal salts such as calcium carbonate, calcium hydroxide, calcium lactate, calcium glycerophosphate, calcium acetate, magnesium carbonate, magnesium hydroxide, magnesium silicate, magnesium aluminate, aluminum hydroxide or aluminum magnesium hydroxide. A particular alkali earth metal salt useful for making an antacid tablet is calcium carbonate.

An example of a low density alkali earth metal salt useful for making the granules according to the present invention is extra light calcium carbonate available from Specialty Minerals Inc., Adams, Me. The density of the extra light calcium carbonate, prior to being processed according to the present invention, is about 0.37 g/ml.

The present invention also relates to administration kits to ease mixing and administration. A month's supply of powder or tablets, for example, can be packaged with a separate month's supply of diluent, and a re-usable plastic dosing cup.

In another embodiment of the present invention, the compositions, methods, combinations, and kits are used as a veterinary preparation, and used alone or combined with an anti-scours agent. An anti-scours agent is a composition which is known to be useful in preventing or inhibiting the symptoms of scours. Known compositions include, for example, colostral extracts from bovine, sheep, goat or other mammals, such as those described in U.S. Pat. No. 4,377,569 and Canadian patent No. 1,175,352 and widely commercially available (for example, Soluble Colostrum Powder, by VedCo, Inc., St. Joseph Mo.; Colostrum Bolus II, by RX Veterinary Products, Kansas City Mo., etc.); an immunological preparation of colostrum isolated from milk-producing mammals which may have been immunized against certain diarrheal causing microorganisms, such as those described in U.S. Pat. No. 4,834,974, Australian patent No. 39340/89, Australian patent No. 52547/90, and German patent No. 1,560,344; microorganism specific immunological preparations, including microorganism specific hybridoma-derived monoclonal antibodies such as those described in Sherman et al., Infection and Immunity, Vol. 42 (2), P. 653-658 (1983) and a bovine immunoglobulin fraction prepared from bovine plasma or clear bovine serum such as the fraction described in U.S. Pat. No. 3,984,539; oral rehydration fluids and/or replacement electrolyte compositions which are widely commercially available in the form of dry compositions or liquid solutions prepared for oral or intravenous administration (for example Electrolyte H, by Agri-Pet Inc., Aubrey Tex.; Electrolyte Powder 8+, by Phoenix Pharmaceutical Inc, St. Joseph Mo.; Electrolyte Solution Rx, by Lextron Inc., Greeley Colo., ProLabs LTD, St. Joseph Mo., and VetTek Inc., Blue Springs Mo.; Calf Rehydrate, by Durvet Inc., Blue Springs Mo., etc.) and antibiotic compositions which are commercially available (for example Biosol®. Liquid, by The UpJohn Company Animal Health Division, Kalamazoo Mich.; Amoxi-Bol®, by SmithKline-Beecham Animal Health, Exton Pa.; 5-Way Calf Scour Bolus™, by Agri Laboratories LTD, St. Joseph Mo.; 1-A-Day Calf Scour Bolus, by A. H. A.; Garacin®. Pig Pump, by Schering-Plough Animal Health Corporation, Kenilworth N.J., etc.).

In one embodiment, the veterinary preparation is a dry preparation of a therapeutic agent of the present invention and an anti-scours agent. The dry preparation may be administered directly or may be hydrated and/or diluted in a liquid solution prior to administration. In another embodiment, the veterinary preparation is a liquid solution of a therapeutic agent of the present invention and an anti-scours agent.

In one embodiment of the present invention, an administration vehicle (for example, pill, tablet, bolus, powder or solution for dilution, pig pump, implant, injectable solution, etc.) contains both a therapeutic agent of the present invention and an anti-diarrheal or anti-scours agent. Thus, the present invention provides pharmaceutical or veterinary compositions, for medical or veterinary use, which comprise a therapeutic agent of the present invention together with at least one pharmaceutically acceptable carriers thereof and other therapeutic ingredients.

The time of administration of a therapeutic agent of the present invention varies depending upon the purpose of the administration. When the compounds of the present invention are administered in order to prevent the development of diarrhea in a subject traveling to areas with high risk of exposure to infectious agent or subjects otherwise exposed to diarrhea causing agents, the compounds should be administered prior to, or at about the time that the subject is exposed to the risk or the high risk area. When the compounds are administered to subjects in order to prevent the development of scours, the therapeutic agent should be administered within the first 12 hours after birth, and preferably within the first 4 hours after birth. When the compounds of the present invention are used to treat subjects having symptoms of diarrhea or scours, the compounds may be administered at any point while the subject is experiencing symptoms, and as soon as the symptoms develop.

In another embodiment, the therapeutic agents come in the form of kits or packages containing a rotavirus-inhibiting agent, for example, a tormentil root extract, and at least one electrolyte, including for example, sodium chloride. Illustratively, the kits or packages contain tormentil root extract and an electrolyte and optionally, a pharmaceutical suitable for diarrhea, in amounts sufficient for the proper dosing of the drugs. In another embodiment, the kits contain a tormentil root extract in a dosage form suitable for oral administration, for example, a tablet or capsule, and an electrolyte in a dosage form suitable for intravenous administration. The therapeutic agents of the present invention can be packaged in the form of kits or packages in which the daily (or other periodic) dosages are arranged for proper sequential or simultaneous administration. The present invention further provides a kit or package containing a plurality of dosage units, adapted for successive daily administration, each dosage unit comprising at least one of the therapeutic agents of the present invention. This drug delivery system can be used to facilitate administering any of the various embodiments of the therapeutic compositions. In one embodiment, the system contains a plurality of dosages to be taken daily via oral administration (as commonly practiced in the oral contraceptive art). In another embodiment, the system contains a plurality of dosages to be administered weekly via transdermal administration (as commonly practiced in the hormone replacement art). In yet another embodiment, the system contains a plurality of dosages to be administered daily, or weekly, or monthly, for example, with at least one therapeutic agent administered orally, and/or at least one therapeutic agent administered intravenously.

The present invention is further illustrated by the following example, which should not be construed as limiting in any way.

### EXAMPLE

### Example 1

Forty children ranging in age from 3 months to 7 years, diagnosed with rotaviral-induced diarrhea, were hospitalized consecutively between February and May 2001 at the Children's Hospital for Infectious Diseases #3 in St. Petersburg, Russia. Parental consent for each child was obtained prior to his or her inclusion into the study. The inclusion criteria were: the period between the beginning of diarrhea and hospitalization was less than 48 hours; the presence of diarrhea when diarrhea was defined as “stool output greater than 10 milliliters per kilogram of body mass per day;” and the stool on admission was positive for RV antigen. The exclusion criteria were: duration of diarrhea over 48 hours; serious somatic pathology; and known allergies to any drugs or foods.

Body mass (without clothing) was recorded by electronic scales within 10 grams on admission and at discharge. The degree of dehydration was determined clinically for each patient on admission and recorded on a scale from 1 to 3:1 for mild, or less than 5%; 2 for moderate, or 5% to 9%; and 3 for severe, or 10% and more. The first stool obtained from each patient admitted to the hospital was analyzed with Immunocard Rotavirus (Meridian Diagnostics, Inc., Cincinnati, Ohio) for rotavirus antigen. Stool output was recorded on a daily basis by weighing diapers for younger children and receptacles for the older children, and then comparing with the dry weight. For younger children, urine was separated from stool with the use of urine collection bags.

The treatment protocol included oral and/or parenteral rehydration, age appropriate enteral feeding as tolerated, and antipyretics as needed. For oral rehydration, Rehydron (Orion Pharma International, Finland) solution was administrated and prepared according to the manufacturer's instructions. Parenteral rehydration solution consisted of glucose, sodium chloride and potassium chloride. Oral rehydration and parenteral rehydration therapy were performed in accordance with World Health Organization recommendations.

After the initial rehydration, the 40 children were randomized into two groups using a computer generated number. The treatment group (20 patients; 12 males and 8 females [age range 4 to 79 months, mean age 24.2±17.2 months]) received tormentil root extract. The control group (20 patients; 13 males and 7 females [age range 3 to 60 months, mean age 24.6±14.0 months]) received a placebo. Tormentil root extract was prepared as an alcohol extract at the hospital pharmacy by taking 100 g of dry rhizome and mixing it with 1000 ml of 70% ethanol. The tormentil was extracted at room temperature with mixing over 7 days. The extract contained 8-10% dry root. The placebo was prepared from a mixture of Indian teas with Hibiscus, which was identical in appearance and taste to tormentil root extract. A bottle containing 8 milliliters of either tormentil root extract or the placebo was prepared for each patient. The research coordinator involved in our double blind study recorded the distribution of tormentil root extract and the placebo used for each patient. A nurse subsequently administered either tormentil root extract or placebo orally, 2 drops per year of the child's life, 3 times daily, beginning the nearest morning. For example, patients admitted in the early afternoon began their trials 20 hours after admission. All patients remained hospitalized until their diarrhea ceased, or until stool output was less than 10 ml/kg/day; stool consistency was normalized; and the symptoms of dehydration were corrected. The following parameters were documented for each patient:

(1) frequency of vomiting and bowel movements;

(2) duration of diarrhea, from hospital admission day until the last day when stool output exceeded 10/ml/kg/day;

(3) duration of vomiting;

(4) daily volume of vomit;

(5) daily urine output;

(6) amount of consumed food; and

(7) volume of liquid received either orally or parenterally.

All children underwent routine laboratory testing throughout the trial, including CBC, serum electrolytes, BUN, creatinine, glucose, SGOT and SGPT. In addition, children's' stool specimens were sent to the laboratory for bacterial cultures.

Significance between the groups was assessed using Student t-test; p value less than 0.05 was considered significant.

All 40 children completed the study. In the tormentil root extract treatment group, no clinical side effects or abnormal laboratory results were detected or noted at follow-up. At the time of hospitalization, children in both groups were similar in age, weight, the degree of dehydration and duration of illness (Table No. 1).

TABLE No. 1
Characteristics of treatment and control groups

| | Treatment group | Control group | P |
|---|---|---|---|
| Age (months) | 24.2 ± 17.2 | 24.6 ± 14.0 | 0.936 |
| Boys (%) | 65 | 60 | 0.751 |
| Weight on admission | 11,974 ± 3,591 | 12,575 ± 3,256 | 0.582 |
| (grams) | | | |
| Duration of fever prior to | 1.1 ± 0.3 | 1.2 ± 0.4 | 0.394 |
| admission (days) | | | |
| Duration of diarrhea prior | 1.6 ± 0.5 | 1.4 ± 0.5 | 0.223 |
| to admission (days) | | | |
| Duration of vomiting prior | 1.3 ± 0.5 | 1.2 ± 0.4 | 0.482 |
| to admission (days) | | | |
| Degree of dehydration on | 1.6 ± 0.5 | 1.5 ± 0.5 | 0.350 |
| admission | | | |

Children in both groups had similar stool and vomit output on the first day, see Table No. 2, below.

TABLE No. 2
Comparison of clinical course in treatment and control groups

| | Treatment group | Control group | p |
|---|---|---|---|
| Correction of dehydration | 1.5 ± 0.5 | 2.25 ± 0.18 | 0.001 |
| (days) | | | |
| Stool output (ml/kg/day) | | | |
| 1 day | 46.8 ± 10.1 | 43.6 ± 8.9 | 0.295 |
| 2 day | 25.9 ± 7.9 | 31.5 ± 7.6 | 0.029 |
| 3 day | 10.9 ± 4.6 | 23.5 ± 7.1 | 0.000 |
| 4 day | 2.7 ± 3.9 | 16.7 ± 4.9 | 0.000 |
| Duration of diarrhea (days) | 2.9 ± 0.9 | 4.8 ± 1.5 | 0.000 |
| Duration of abnormal stool | 3.5 ± .5 | 5.9 ± 1.5 | 0.000 |
| consistency (days) | | | |
| Volume of Vomit | | | |
| (ml/kg/day) | | | |
| 1 day | 14.2 ± 3.8 | 13.9 ± 0.5 | 0.846 |
| 2 day | 4.1 ± 4.6 | 5.0 ± 5.6 | 0.588 |
| Duration of vomiting after | 1.6 ± 0.5 | 1.5 ± 0.5 | 0.350 |
| admission (days) | | | |
| Oral rehydration volume | | | |
| (ml/kg/day) | | | |
| 1 day | 55.5 ± 24.1 | 57.1 ± 17.4 | 0.817 |
| 2 day | 67.3 ± 28.1 | 63.8 ± 26.6 | 0.684 |
| Intravenous fluids volume | | | |
| (ml/kg/day) | | | |
| 1 day | 40.7 ± 21.9 | 45.9 ± 21.8 | 0.457 |
| 2 day | 22.9 ± 24.2 | 39.5 ± 21.1 | 0.026 |
| Enteral feeding volume | | | |
| (ml/kg/day) | | | |
| 1 day | 40.1 ± 21.9 | 38.4 ± 12.9 | 0.736 |
| 2 day | 64.3 ± 27.9 | 58.5 ± 25.9 | 0.504 |
| 3 day | 61.9 ± 31.1 | 20.4 ± 39.1 | 0.000 |
| Discharge weight (grams) | 11,442 ± 3,618 | 12,002 ± 3,150 | 0.604 |

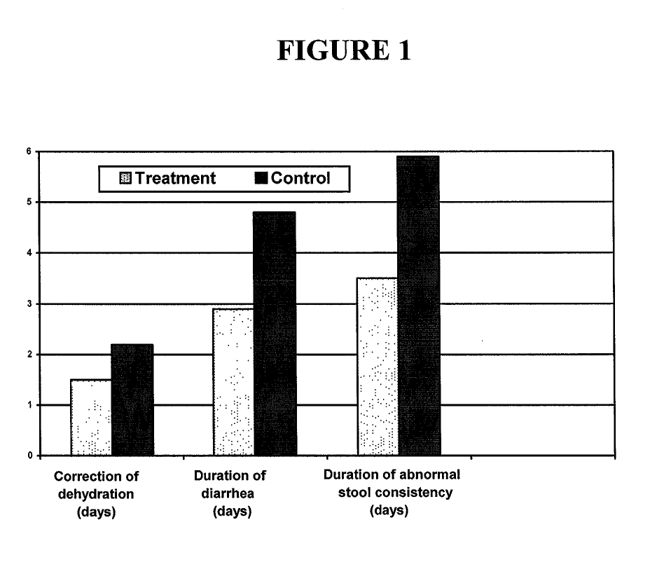

However, there was a statistically significant reduction in duration of diarrhea in the treatment group compared to the control group (2.9 days vs. 4.8 days) (See FIG. 1). After 48 hours, diarrhea ceased in 8/20 (40%) children who received tormentil root extract, compared to 1 child (5%) in the control group who received the placebo. As a result, required rehydration therapy was less in the treatment group compared to the control group. The volume of solutions for oral rehydration in both groups was comparable on day 1. However, on day 2, patients in the treatment group required less parenteral rehydration solutions than patients in the control group. In addition, stool consistency in children in the treatment group was normalized within 3.5 days of admission, compared to 5.9 days in the placebo group patients. There was no effect on the volume of vomit output or the duration of vomiting. The volume of enteral nutrition was similar in both groups during the first two days; however, by day 3, it was significantly greater in the treatment group.

According to calculations, children in this study who received tormentil root extract received a tannin dose of approximately 0.5 mg/kg/day. This amount of tannins would be unlikely to shorten the duration of diarrhea.

The duration of diarrhea in the tormentil root extract group was shorter than in the control group. It is possible, that earlier administration of tormentil root extract could produce even better results. In fact, it was observed in several cases in which diarrhea ceased following a single dose of tormentil root extract. On the other hand, tormentil root extract did not influence the duration or volume of vomiting, the volume of oral rehydration, or the weight change on admission and on discharge. Nevertheless, the children in the tormentil root extract treatment group required smaller volumes of parenteral rehydration solution and were able to better tolerate enteral nutrition.

Results from this clinical trial demonstrate that tormentil root extract is a safe and effective agent that reduces the amount of fluid loss in rotaviral-induced diarrhea and shortens the duration of diarrhea. It is further contemplated that tormentil root extract will be beneficial in decreasing morbidity and mortality in developing countries as well as decreasing the cost of care in developed countries.

The practice of the present invention will employ, unless otherwise indicated, conventional techniques of pharmacology and pharmaceutics, which are within the skill of the art.

All cited literature and patent references are hereby incorporated herein by reference. Although the invention has been described with respect to specific embodiments and examples, it should be appreciated that other embodiments utilizing the concept of the present invention are possible without departing from the scope of the invention. The claimed elements, and any and all modifications, variations or equivalents that fall within the true spirit and scope of the underlying principles define the present invention.

What is claimed is:

1. A pharmaceutical composition, comprising: an anti-diarrheal agent and an electrolyte, wherein the amount of the agent and the electrolyte together make an anti-diarrhea effective amount.

2. The composition of claim 1, wherein the agent comprises tormentil root extract.

3. The composition of claim 2, wherein the tormentil root extract is present in an amount from about 1 mg to about 200 mg per gram of the composition.

4. The composition of claim 3, wherein the tormentil root extract is present in an amount from about 5 mg to about 100 mg per gram of the composition.

5. The composition of claim 1, wherein the electrolyte is selected from the group consisting of sodium, potassium, and chloride.

6. The composition of claim 5, wherein the electrolyte is present in an amount from about 5 mg to about 950 mg per gram of the composition.

7. The composition of claim 5, wherein the electrolyte is present in an amount from about 100 mg to about 500 mg per gram of the composition.

8. The composition of claim 2, further comprising a tannin.

9. The composition of claim 1, wherein the anti-diarrheal agent is selected from the group consisting of a probiotic, an antiperistaltic agent, an antisecretory agent, an immunoglobulin, diphenoxylate, atropine, hyoscyamine, metoclopramide, an antibiotic, or loperamide.

10. The composition of claim 9, wherein the antibiotic is selected from the group consisting of tetracycline, doxycycline, trirmethoprim, sulfamethoxazole, a penicillin, a cephalosporin, a quinolone, and an aminoglycoside.

11. The composition of claim 10, wherein the quinolone is selected from the group consisting of norfloxacin and ciprofloxacin.

12. The composition of claim 1, wherein the composition is in a dosage form selected from the group consisting of tablet, pill, suspension tablet, powder, lozenge, sachet, cachet, elixir, suspension, emulsion, solution, syrup, aerosol, ointment, soft gelatin capsule, hard gelatin capsule, and suppository.

13. The composition of claim 12, wherein the solution comprises a sterile injectable solution.

14. The composition of claim 12, wherein the dosage form is further selected from the group consisting of immediate release, sustained release, and delayed release.

15. The composition of claim 12, further comprising an agent selected from the group consisting of an excipient, a lubricant, a wetting agent, an emulsifier, a suspending agent, a preservative, and a flavoring agent.

16. The composition of claim 15, wherein the excipient comprises lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, or methyl cellulose.

17. The composition of claim 16, wherein the lubricating agent comprises talc, magnesium stearate, or mineral oil.

18. The composition of claim 2, wherein the root extract comprises its salt, ester, amide, enantiomer, isomer, tautomer, or prodrug forms.

19. The composition of claim 12, wherein the dosage form is a powder comprising about 0.1% to about 95% tormentil root extract weight to weight of the composition.

20. The composition of claim 12, wherein the dosage form is a tablet, suspension tablet, pill, lozenge, sachet, cachet or capsule comprising about 0.1% to about 95% tormentil root extract weight to weight of the composition.

21. The composition of claim 12, wherein the dosage form is an elixir, suspension, emulsion, solution or syrup comprising about 0.1% to about 95% tormentil root extract weight to weight of the composition.

22. A method for treating, preventing or reducing the risk of developing diarrhea in a mammal in need thereof, comprising: administering to the mammal a composition comprising an anti-diarrheal agent and an electrolyte, wherein the amounts of the agent and the electrolyte together make an anti-diarrhea effective amount.

23. The method of claim 22, wherein the agent comprises tormentil root extract.

24. The method of claim 23, wherein the tormentil root extract is administered in an amount of about 0.1 mg/kg to about 10.0 mg/kg body weight per dose up to 6 doses per day.

25. The method of claim 23, wherein the tormentil root extract is administered in an amount of about 1.0 mg/kg to about 5.0 mg/kg body weight per dose up to 6 doses per day.

26. The method of claim 22, wherein the electrolyte is selected from the group consisting of sodium, potassium and chloride.

27. The method of claim 22, wherein the electrolyte is administered in an amount of about 1.0 mg/kg to about 100 mg/kg body weight per dose up to 6 doses per day.

28. The method of claim 22, wherein the electrolyte is administered in an amount of about 5.0 mg/kg to about 20 mg/kg body weight per dose up to 6 doses per day.

29. The method of claim 22, wherein the diarrhea is selected from the group consisting of exudative diarrhea, non-exudative diarrhea, decreased absorption diarrhea, non-decreased absorption diarrhea, inflammatory diarrhea, non-inflammatory diarrhea, secretory diarrhea, and non-secretory diarrhea.

30. The method of claim 22, wherein the composition is in a dosage form selected from the group consisting of tablet, pill, suspension tablet, powder, lozenge, sachet, cachet, elixir, suspension, emulsion, solution, syrup, aerosol, ointment, soft gelatin capsule, hard gelatin capsule, and suppository.

31. The method of claim 23, further comprising administering a tannin in an amount of about 0.5 mg/kg to 6000 mg/kg body weight per dose.

32. A solution, comprising:
(a) about 10 to 1000 mg/L tormentil root extract;
(b) about 10 to 150 mEq/L sodium;
(c) about 1 to 30 mEq/L potassium;
(d) about 10 to 150 mEq/L chloride;
(e) about 10 to 150 mEq/L citrate; and
(f) about 10 to 150 g/L dextrose.
